(19) United States
(12) Patent Application Publication
(10) Pub. No.: US 2003/0228586 A1
(43) Pub. Date: Dec. 11, 2003

(54) Method for measuring de novo T-cell production in humans

(76) Inventors: Rafick-Pierre Sekaly, St. Laurent (CA); Jean-Francois Poulin, Montreal (CA); Morgan Jenkins, San Francisco, CA (US); Jeffrey M. Harris, San Francisco, CA (US); Krishna V. Komanduri, San Francisco, CA (US); Joseph M. McCune, San Francisco, CA (US)

Correspondence Address: Keown & Associates, Suite 1200, 500 West Cummings Park, Woburn, MA, 01801, US

(21) Appl. No.: 10/341,015

(22) Filed: Jan. 13, 2003

Related U.S. Application Data
(63) Continuation of application No. 09/607,908, filed on Jun. 30, 2000, now abandoned.
(60) Provisional application No. 60/141,265, filed on Jun. 30, 1999.

(51) Int. Cl. C12Q 1/68; C12P 19/34

(52) U.S. Cl. 435/6; 435/91.2

(57) ABSTRACT

The present invention relates to a method for measuring de novo T-cell production in humans, and more particularly to the assessement recent thymic emigrant (RTE) diversity in a T-cell sub-population of a patient by the detection of T-cell receptor β chain DNA deletion circles (TCRβDC) generated during TCR gene rearrangement of thymocytes in the thymus. The method comprises isolating a T-cell sub-population from a patient, extracting genomic DNA from the T-cell sub-population, amplifying the genomic DNA with a primer specific for a T-cell receptor β chain DNA rearrangement deletion circle (TCRβDC) family and detecting the TCRβDC, the TRCβDC being indicative of the presence of a RTE. The method assesses the quantitative and qualitative (diversity) intrathymic T-cell production by quantitating the relative frequency and diversity of RTEs within various sub-populations of circulating human T-cells. Such a method may be useful to study the diversity of the human thymic function and to monitor immune reconstitution of HIV patients.

### BACKGROUND OF THE INVENTION

(a) Field of the Invention

The present invention relates to a method for measuring de novo T-cell production in humans.

(b) Description of Prior Art

T-cell progenitors from the lymphoid stem cells emerge from the bone marrow and enter the thymus where they undergo a T-cell receptor (TCR) rearrangement and differentiate into T-cells expressing the CD4 (T-helper) and CD8 (T-cytotoxic) clusters of differentiation antigens, following a selection by the major histocompatibility complex (MHC) class I molecules, which activate T-cytotoxic cells, and class II molecules, which activate T-helper cells. Only 5% of the thymocytes leave the thymus as mature CD4 or CD8 T-cells.

TCRs have a β chain and an α chain. The variable portion of the TCR β chain is formed by the variable region (V), the diversity region (D) and the joining region (J), while the α chain is formed by the variable segment (V) and the joining segment (J). Variable genes that are similar to each other are called “families”. The different genes juxtapose with each other during the process of DNA rearrangement. This leads to the generation of a very vast set of TCR in the order of 10¹⁵. The TCR repertoire comprises all the TCRs expressed within an individual.

Antigen-presenting cells, such as macrophages or infected cells, process viral antigens and display fragments called epitopes on the cell surface in association with molecules of (MHC). The epitope associated with the MHC molecule is then recognized by effector T-cells, which then proliferate, producing billions of cells with the same T-cell receptor. There are monoclonal (all entirely the same) and oligoclonal (a few T-cell receptors) expansions.

The number of cells expressing the different families of TCRs remains stable throughout life, but can vary upon exposure to an infectious agent.

It has been assumed that a diverse TCR repertoire is formed during early life, when the thymus is most active, and that T-cell homeostasis is maintained without significant thymic input in adults (Mackall, et al. (1997) Immunol. Today 18:245-251 “T-cell Regeneration: All Repertoires are not Created Equal”; McCune, J. M. (1997) Sem. Immunol. 9:397-404 “Thymic Function in HIV-1 Disease).

Given the profound effects of stress upon thymopoiesis, intrathymic T-cell production in an intact animal is best studied with a minimally invasive assay for recent thymic emigrants (RTES) in the peripheral blood.

For example, RTEs can be identified in the chicken by their unique expression of the chT1 cell-surface marker (Kong, et als. (1998) Immunity 8:97-104 “Thymic function can be accurately monitored by the level of recent T cell emigrants in the circulation”). Murine RTEs may be followed kinetically in the peripheral circulation after direct intrathymic labeling such as with fluorescein isothiocyanate Scollay, et als. (1980) Eur. J. ImmunoL 10:210-218 “Thymus cell migration. Quantitative aspects of cellular traffic from the thymus to the periphery in mice.”

Assays of this type are, however, unavailable for the assessment of human thymic function since no specific cell-surface marker for human RTEs has been identified. Such assessment has relied instead upon autopsy series (Steinmann, G. G. (1986) Histopathology and Pathology (Muller-Hermelink H. K., ed) Springer, New York, pp. 43-48 (1986) “Changes in the human thymus during aging, in The Human Thymus”) radiographic observations (Francis, et als. (1985) Am. J. Radiol. 145:249-254 “The thymus: Reexamination of age-related changes in size and shape) and/or phenotypic demarcation of circulating human T-cells into distinct populations of “naive” or “memory/effector” cells (Picker, et als. (1993) J. Immunol. 150:1105-1121 “Control of lymphocyte recirculation in man. 1. Differential regulation of the peripheral lymph node homing receptor L-selection on T cells during the virgin to memory cell transition”) Five-color flow cytometry may be used to distinguish memory from naïve T-cells by detecting the CD45RO cell-surface marker for the memory T-cells and the CD45RA and L-selectin (CD62L) cell-surface markers for the naïve T-cells. The simultaneous expression of CD45RA and L-selectin on the cell surface indicates that a cell is a recently generated thymic emigrant (RTE) heading towards a lymph node.

These studies demonstrate that (a) there is a correlation between the abundance of circulating CD4⁺CD45RA⁺CD62L⁺ (naive) human T-cells and the presence of thymic tissues (Picker, et als. (1993) J. Immunol. 150:1105-1121 “Control of lymphocyte recirculation in man. 1. Differential regulation of the peripheral lymph node homing receptor L-selection on T cells during the virgin to memory cell transition; Heitger, et als. (1997) Blood 90:850-857 “Essential role of the thymus to reconstitute naive (CD45RA+) T-helper cells after human allogeneic bone marrow transplantation; McCune, et als (1998) J. Clin. Invest. 101:2301-2308 “High prevalence of thymic tissue in adults with human immunodeficiency virus-1 infection,” suggesting that RTEs are included within this T-cell sub-population; (b) the circulating CD8⁺CD45RA⁺T-cell sub-population is less clearly associated with human thymic tissue (Heitger, et als. (1997) Blood 90:850-857 “Essential role of the thymus to reconstitute naive (CD45RA+) T-helper cells after human allogeneic bone marrow transplantation) and (c) circulating “memory/effector” CD4⁺ and CD8⁺ T-cell sub-populations bear the phenotypic marker CD45RO instead of CD45RA (Sanders, et als. (1988) Immunol. Today. 9:195-199 “Human naive and memory T cells”).

However, phenotypic measures are imprecise in their ability to distinguish lymphocytes which have recently differentiated in the thymus or peripheral tissues and those which have reverted from memory status (Tough, D F, et al. (1995) Stem Cells. 13:242-249 “Life span of naive and memory T cells;” Bell, E. B., et al. (1990) Nature 348:163-166 “Interconversion of CD45R subsets of CD4 T cells in vivo”). The inconsistencies reported in studies relying on these measures may be attributable to their failure to distinguish this group of cells. Thus, although it is clear that the human thymus involutes dramatically after puberty (Steinmann, G. G. (1986) Histopathology and Pathology (Muller-Hermelink H. K., ed) Springer, New York, pp. 43-48, “Changes in the human thymus during aging, in The Human Thymus”), the fraction of circulating CD45RA⁺ T-cells remains relatively constant for long periods of time thereafter (Erkeller-Yuksel, et als. (1992) J. Pediatr. 120:216-222 “Age-related changes in human blood lymphocyte subpopulations). These findings suggest that the CD45RA⁺CD62L⁺ T-cell sub-population may contain a higher proportion of RTEs earlier than later in life, and that it harbors heterogeneous cell populations, including revertants of memory/effector cells.

An intrinsic feature of the TCR rearrangement process has been exploited to directly demonstrate the presence of continuous thymic output in human adults (Douek, et als. (1998) Nature 396:690-695 “Changes in thymic function with age and during the treatment of HIV infection). This assay relies on the detection of TCR α excision circles (αTRECs) generated during TCR α gene rearrangement in the thymus. Similar observations were also made in the avian system whereby de novo TCR rearrangement, as measured by excision circle assays, correlated with the expression of the chT1 antigen (Kong, F., et als. (1998) Immunity 1:97-104 “Thymic function can be accurately monitored by the level of recent T cell emigrants in the circulation”). Moreover, circle-bearing T-cells were found in the avian lymph node, spleen and skin (Kong, F. K., et als. (1999) Proc. Nati. Acad Sci. (U.S.A.) 96:1536-1540 “T cell receptor gene deletion circles identify recent thymic emigrants in the peripheral T cell pool”), suggesting that the thymus may constantly supply new T-cells to these peripheral compartments.

The thymus is well accepted as being the primary site of thymopoiesis, even if some recent reports suggested the existence of thymic-independent T-cell generation pathways. However, the contribution of the bone marrow and gut-associated lymphoid tissues to the overall de novo T-cell production is still unknown. These extra-thymic compartments may act as “backup systems” in case of need (when the thymus cannot compensate a massive peripheral T-cell depletion by itself) Human T-cell homeostasis has often been studied with the use of proliferation (Ki67 and BrdU) and cell-surface (CD45RA, CD45RO and CD62L) markers. However, the regulation of these markers is not completely known T-cell compartments are highly heterogeneous and complexly interconnected, more specialized tools that would be generated could deepen our comprehension regarding the life span of T-cells. The immune system homeostasis will be understood only when complete characterization of T-cell input/output sources will be done.

In a patient infected with a virus such as HIV, naive T-cells progressively disappear from the peripheral blood, while memory T-cells accumulate. However, the proportion of memory and naive T-cells is relatively stable during HAART (highly active antiretroviral therapy) treatment. Following HAART, there is an initial increase in the proportion of memory T-cells, which is followed by a gradual increase in the absolute and relative numbers of naive T-cells. The inversion of the naive-to-memory ratio may be the result of an accumulation of RTEs that renew the T-cell pool. It would therefore be useful to have a method for uncovering new T-cell gene rearrangement.

There are at present no assays available for the assessment of the diversity of human thymic function and more particularly the diversity of RTEs based on a specific marker.

It would, therefore, be highly desirable to be provided with a method for detecting RTEs within various sub-populations of circulating human T-cells.

It would also be highly desirable to be provided with a method for assessing the overall de novo T-cell production in humans.

### SUMMARY OF THE INVENTION

One aim of the present invention is to provide a method to assess the quantitative and qualitative (diversity) intrathymic T-cell production by quantitating the relative frequency and diversity of recent thymic emigrants (RTEs) in the peripheral blood of humans and more particularly within various sub-populations of circulating human T-cells.

Another aim of the present invention is to provide monoclonal antibodies (mAbs) to detect recent thymic emigrant (RTE) diversity in a patient. The method of the present invention may be used to develop a biological reagent which would be used to identify this subset.

In accordance with the present invention there is provided a method for detecting recent thymic emigrant (RTE) diversity in a T-cell sub-population of a patient. The method comprises isolating a T-cell sub-population from a patient, extracting genomic DNA from the T-cell sub-population, amplifying the genomic DNA with a primer specific for a T-cell receptor β chain DNA rearrangement deletion circle (TCRβDC) family and detecting the TCRβDC, the TRCβDC being indicative of the presence of a RTE.

The extracted genomic DNA may be diluted prior to the amplification.

The amplification may be effected with a polymerase chain reaction (PCR).

The extracted genomic DNA may be spectrophotometrically quantitated to detect the TCRβDC prior to the dilution.

The dilution may be effected 4 or 5 folds.

A dilution endpoint of TCRβDC may be determined for the dilution.

A positive signal corresponding to an endpoint may be detected at a highest dilution, and a TCRβDC 50% endpoint and a TCRβDC frequency may be determined.

The endpoint may be calculated with a Reed-Muench method or a maximum likelihood estimate.

The extracted total genomic DNA may be amplified a first time with a Dβ-specific primer and a Vβ-specific primer, and the amplified DNA may be amplified a second time with a nested primer.

The TCRβDC may be detected with an agarose gel electrophoresis under a UV light.

The primer may have a sequence selected from the group consisting of SEQ ID NO:1, SEQ ID NO:2, SEQ ID NO:3, SEQ ID NO:4, SEQ ID NO:5, SEQ ID NO:6 and SEQ ID NO:7.

The T-cell sub-population may be selected from the group consisting of CD3⁺CD8⁺ thymocytes, CD4⁺CD8⁺ thymocytes, CD3⁺CD4⁺CD8⁻ thymocytes, CD3+CD4^{-CD}8⁺ thymocytes, CD4⁺CD45RA⁺CD62^{L+} lymphocytes, CD4⁺CD45RA⁺CD62L⁻ lymphocytes, CD4⁺CD45RO⁺CD62^{L+} lymphocytes, CD4⁺CD45RO⁺CD62L⁻ lymphocytes and CD4^{+CD}56RO⁻CD62L⁺ lymphocytes.

The T-cell sub-population may be isolated from a peripheral blood sample, a cord blood sample or a tissue section collected from said patient.

The amplified TRCβDC family may consist of a Vβ/Dβ family.

The primer may be specific for a Vβ2/Dβ1, Vβ5.1/Dβ1, Vβ9/Dβ1, Vβ14/Dβ1, Vβ16/Dβ1, Vβ17/Dβ1 or Vβ22/Dβ1 DC family.

The specifice primer may be used with TaqMan.

The cell-surface marker may comprise CD45RA and CD62L.

The patient may be infected with HIV or may have undergone a myeloablation.

The T-cell sub-population may be isolated from said peripheral blood sample by staining said peripheral blood sample with fluorescent-conjugated monoclonal antibodies specific for a cell-surface marker.

The T-cell sub-population may be isolated by flow cytometry.

The T-cell sub-population may be isolated by cell sort-purification.

The genomic DNA may be recovered with a proteinase K.

In accordance with yet another aspect of the invention, there is provided a method for developing monoclonal antibodies (mAbs) to identify a recent thymic emigrant population in a patient. A panel of cell-surface markers, such as of the conventional type, may be used to delineate the subset of T-cells enriched in DCs. The CD45RA⁺CD62L⁺ cells may be further subdivided into many different subsets. These subsets may be sorted and tested for the presence of DCs using the method of the present invention. Once the subset is identified, cells or plasma membranes isolated from the equivalent of 10⁷ cells may be used to immunize Ba1b-C mice. Three weeks after initial immunization, the mice may be subjected to two different boosts each with the same number of cells. The mice may be sacrificed and spleen cells therefrom may be fused with a B-cell lymphoma fusion partner using polyethylene glycol. Selection of hybridomas may be carried out using appropriate selection markers. Screening of supernatants from hybrids between spleen cells and the fusion partner may be carried out using multiple color flow cytometry. In particular, antibodies who can identify restricted subsets within the CD45RA⁺CD62L⁺ cells may be detected. Once such antibodies have been isolated, cells may be sorted and it may be verified that they are exclusive for cells which carry DCs.

In accordance with yet another aspect of the invention, there is provided a method for detecting T-cell receptor β chain DNA rearrangement deletion circles (TCRβDC) in a cell population from a patient. The method comprises isolating a cell population from a patient, extracting genomic DNA from the cell population, diluting the extracted DNA, amplifying the diluted DNA with a primer specific for a TCRβDC family and effecting an endpoint dilution analysis of the amplified DNA for the TCRβDC family.

In accordance with yet another aspect of the invention, there is provided a method for detecting T-cell receptor β chain DNA deletion circles (TCRβDC) in a T-cell. The method comprises amplifying a genomic DNA of the cell with a primer specific for a TCRβDC family and detecting the amplified DNA indicative of a newly generated T-cell.

### BRIEF DESCRIPTION OF THE DRAWINGS

Having thus generally described the nature of the present invention, reference will now be made to the accompanying drawings, showing by way of illustration a preferred embodiment thereof, and in which:

FIG. 1 illustrates the formation and detection of TCRβ rearrangement deletion circles (DCs); 1A (top) shows the genomic organization of the region including the Vβ2 and Dβ1 coding segments, flanked by heptamer and nonamer recombination signal sequences (RSSs) and 170 kbp of intervening noncoding DNA and (bottom) the generation of a rearranged Vβ2/Dβ1 coding TCR and a 170 kbp Vβ2/Dβ1 deletion circle after excision-ligation mediated by the recombination activation genes RAG-1 and RAG-2; the relative location and orientation of the primers used for amplification of the unique signal joint are shown; note that DCs have various sizes (from 65 kbp to 588 kbp) depending on the Vβ-Dβ usage; the Vβ (variable region), Dβ (diversity region) and Jβ (joining region) coding genes segments rearrange first between the Dβ gene segment and the Jβ gene segment, and then between the Vβ gene segment and the rearranged Dβ-Jβ gene segment to form the coding sequence of TCR variable region; 1B (top) shows a map of the amplified 439 bp Vβ2/Dβ PCR product; (bottom) shows a representative example of Vβ2/Dβ1 DC products amplified from CD4⁺CD8⁺ human thymocytes or from Jurkat cells; the left gel shows the specificity of the amplification; note the absence of products in both the Jurkat and “no DNA” lanes; the PCR product is partially cleavable by ApaL1, likely due to heterogeneity of nucleotide sequence at the circle junction; an ApaL1 digestion positive-control was performed at the same time on an empty pBS vector, resulting in complete digestion; the right gel shows restriction analysis of the purified 439 bp Vβ2/Dβ1 DC product, with characteristic cuts by Sac1, Pvu11, and ApaL1; the white arrow points at the 55 bp fragment released by ApaL1 digestion;

FIG. 2 illustrates the quantitation of TCR rearrangement DCs; 2A shows a representative example of endpoint dilution analysis of DC within CD3⁺CD8⁺ human thymocytes; starting at 2000 μg of input DNA per well, quadruplicate 5-fold serial dilutions were subjected to the nested PCR approach shown in FIG. 1; DNA from Jurkat cells (150 μg) and from total thymocytes (150 μg) served as negative and positive controls, respectively; 2B shows the relative frequencies of Vβ2/Dβ1 DC in sort-purified populations of CD4⁺CD8⁺, CD3⁺CD4⁺CD8⁻ and CD3⁺CD4⁻CD8⁺human thymocytes; and

FIG. 3 illustrates the detection of TCR rearrangement DCs in human peripheral blood T-cells; 3A shows the representative flow cytograms of CD4⁺ human cord blood T-cells that were unstimulated (panel 1) or stimulated for varying time intervals (panel 2: 72 hr, panel 3: 96 hr, panel 4: 9 days) with IL-2 (10 U/mL) and PHA (5 ug/mL); CD4⁺ T-cells at each time point were gated and subdivided by staining for CD45RA and CD62L markers; based on the staining of cells for CD45RA before stimulation (panel 1), cells were designated as CD45RA^{Bright} or CD45RA^{Dim} (with fluorescence intensities above and below the dotted lines, respectively); 3B shows the relative frequency of Vβ2/Dβ1 DCs in cord blood T-cells that were unstimulated (control) or stimulated for varying time intervals with PHA and IL-2; the black bars show results from one experiment with endpoints at 48 hr and 72 hr; the white bars show results from a second experiment (different cord blood donor) with endpoints at 72 hr and 9 days; 3C shows the correlation between increasing age and decreasing frequency of Vβ2/Dβ1 DCs in the circulating CD4⁺CD45RA⁺CD62L⁺ T-cell sub-population (p=0.0045); sort-purified CD4⁺CD45RA⁺CD62L⁺ human peripheral blood T-cells were isolated from individuals of the indicated ages and analyzed for Vβ2/Dβ1 DCs; such DCs were absent from the CD4⁺CD45RO⁺CD62L⁻ sub-populations of each individual (not shown); the point at 55 years old was scored as “undetectable” in the assay (i.e. with a DCF value of 0.1 or less); and 3D shows the percentages of circulating naive (CD45RA⁺CD62L⁺) CD4⁺ T-cells in the peripheral blood as a function of age; no correlation exists between the age and the frequency of such naive CD4⁺ T-cells (p=0.5123).

### DETAILED DESCRIPTION OF THE INVENTION

In accordance with the present invention, there is provided a method to quantitate the relative frequency and diversity of recent thymic emigrants (RTEs) in the peripheral blood of a patient. There is provided a PCR-based assay to evaluate the relative frequency of RTEs in the peripheral blood of human patients and more particularly adults. Such an assay aims at detecting DNA deletion circles (DCs), which are by-products of TCR gene recombination (Roth, et als. (1992) Cell 70:983-991 “V(D)J recombination: broken DNA molecules with covalently sealed (hairpin) coding ends in SCID mouse thymocytes;” and Kong, et als. (1998) Immunity 8:97-104 “Thymic function can be accurately monitored by the level of recent T cell emigrants in the circulation”). This non-invasive method directly demonstrates the contribution of new T-cells to the peripheral circulation and provides, for the first time, a measure of de novo T-cell production in humans.

Recent thymic emigrants (RTEs) cells are detected by the presence of TCR rearrangement deletion circles (DCs) and episomal by-products of the TCRβ V, D, and J rearrangement within them.

RTEs are most abundant in the CD45RA⁺CD62L⁺ sub-population, are at least oligoclonal in their expression of TCR Vβ regions and are detectable in adults.

Deletion circles (DCs) were detected in T-cells in the thymus, in cord blood, and in adult peripheral blood. In the peripheral blood of adults aged 22 to 76 years, the DCs frequency is highest in the CD4⁺CD45RA⁺CD62L⁺ sub-population of naive T-cells TCR DCs are also observed in other sub-populations of peripheral blood T-cells, including those with the CD4⁺CD45RO⁻CD62L⁺ and CD4⁺CD45RO⁺CD62L⁺ phenotype. RTEs were observed to have more than one rearrangement, suggesting that replenishment of the repertoire in the adult is at least oligoclonal. These results demonstrate that the normal adult thymus continues to contribute, even at old ages, a diverse set of new T-cells to the peripheral circulation.

The method of the present invention allows the detection and quantification of the relative frequency of DCs in T-cell populations, thereby evaluating de novo T-cell production levels.

PCR amplification of a given VβDβ DC family is also possible with the use of specific primers, hybridizing to unique DNA sequences (Okazaki, et al. (1987) Cell 49:477-485 “T cell Receptor p Gene Sequences in the Circular DNA of Thymocyte Nuclei: Direct Evidence for Intramolecular DNA Deletion in V-D-J Joining”). This ability to discriminate and evaluate the relative frequency of any DC family allows one to establish the breadth of the newly generated T-cell repertoire, e.g., restricted or unrestricted to several Vβs. This application may have a tremendous effect on various fields of research, notably on immune reconstitution and T-cell repertoire studies.

The first version of the deletion circle assay, involving replicate dilution series of DNA from sort-purified cell sub-populations, was costly (a few thousand dollars per sample), time consuming (taking an average of 2-3 days to finish), and dependent upon the use of expensive equipment and reagents (including a fluorescence-activated cell sorter, a thermocycler for running polymerase chain reaction (PCR) assays, and associated reagents such as fluoresceinated antibodies and TAQ polymerase).

The semi-quantitative assay of the present invention aims at measuring the dilution endpoint of DNA deletion circles. Total genomic DNA from a sorted T-cell population is serially diluted and four PCR replicate series are carried out to determine whether a given well is positive or negative for the DCs. The “50% DC endpoint,” measured in terms of nanograms of input DNA, is calculated using either the Reed-Muench method (Rabin, L., et als. (1996) Antimicrob. Agents Chemother. 40:755-762 “Use of standardized SCID-hu Thy/Liv mouse model for preclinical efficacy testing of anti-human immunodeficiency virus type 1 compounds;” and Ausubel, F. M. et al. (1987) Interscience, New York, pp. 2.2.1-2.2.3 Current Protocols in Molecular Biology, “Preparation of genomic DNA from mammalian tissue”) or a maximum likelihood estimate (Rowen, L., et als. (1996) Science 272:1755-1762, “The complete 685-kilobase DNA sequence of the human β T cell receptor locus”). The 50% DC endpoint represents the median minimal amount of DNA from which a deletion circle may be amplified by semi-nested PCR. This “50% PC endpoint” allows comparison between different T-cell populations. The assay of the present invention is currently being optimized to generate quantitative answers, e.g. the number of copies of a given DC family per 10⁵ sorted T-cells. The following assays for DCs are being developed, each of which may be easier to perform, less expensive, and more quantitative: (1) A quantitative, real-time polymerase chain reaction (PCR) for DCs using the “TAQman” methodology and primers specific for TCR Vβ DC, which is capable of detecting and quantitating DC DNA within unseparated human peripheral blood mononuclear cells (PBMCs), thereby minimizing the need for cell sorting. This assay provides information about the absolute number of DCs within a given cell sample; and (2) a polymerase chain reaction/in situ hybridization for DC for analysis of DC DNA at the single-cell level, using the polymerase chain reaction and primers specific for TCR Vβ DC to directly amplify DC within single cells; ISH (using enzymatic, fluorescent or radioactive detection) is then used to identify the amplified products. This approach may be amenable to the analysis of DC DNA within single cells in tissue sections and/or by flow cytometry.

The DC assay may be applicable to important contemporary questions about the diversity of the thymic function and immune reconstitution in humans. Most immediately, it may be of interest to determine whether and under which circumstances thymic function may be present in patients with advanced HIV disease or post-myeloablation. This measure of thymic function may also facilitate the design of studies aimed at augmenting intrathymic T-cell production

The method of the present invention detects physical evidence of recent TCR gene rearrangement, within adult human peripheral blood mononuclear cells (PBMC), of TCR 62 DNA deletion circles, a characteristic of recently rearranged T-cells. This noninvasive method may therefore be used to monitor de novo T-cell production in humans.

This semi-quantitative assay may be optimized in such a way that strong quantitative statements such as the number of DNA DCs present in 1 μg of total genomic DNA and the percentage of recently rearranged T-cells within the CD4-expressing T-cell population may be made. A competitive PCR assay may be obtained which is more scientific and “user friendly”. Fluorescence in situ hybridization (using a fluorescent DNA DC probe) is also contemplated.

Depending on the Vβ/Dβ TCR usage, distinct DNA DCs are generated. All potential DNA DCs range from 65 kbp to 590 kbp and can be recovered using a simple and straightforward Proteinase K-based total genomic DNA isolation procedure. This protocol yields approximately 1 μg of purified genomic DNA per 100,000-150,000 cells (either thymocytes, cord blood mononuclear cells (CBMC) or peripheral blood mononuclear cells (PBMC).

In a preferred embodiment, the purified genomic DNA is quantified at 260 nm and 280 nm, serially diluted (5-fold dilutions), and thermal cycling is performed in quadruplicates on each of the dilutions to ensure a precise end-point read-out for each experiment. First-round PCR amplification necessitates the DC-Dβ1 primer with any of the DC-Vβ specific primers. From the first amplification, 3 μL is used as a template for the semi-nested PCR. Second round PCR is performed in identical conditions using a CIRCLE-Vβ specific primer (nested primer) instead of the DC-Vβ specific primer (outer primer) Second round PCR products are visualized under ultraviolet lights following agarose gel electrophoresis. Each dilution of all 4 replicates is scored positive or negative by two observers and a “50% endpoint” is calculated using the method described by Reed and Muench. The 50% endpoint corresponds to the amount of total genomic DNA needed to give rise to a deletion circle specific signal 50% of the time. This non-parametric analysis allows to quantitate the relative frequency of DNA deletion circle found in a given sorted cell population compared to another.

The method of the present invention enables the determination of the relative frequency of newly produced T-cells in the peripheral circulation of adult humans and the presence/absence of DCs within them. The method of the present invention is independent of cell-surface marker expression and may enlighten the understanding of thymic function and T-cell homeostasis.

To determine whether the CD4⁺CD45RA⁺CD62L⁺ sub-population of circulating human T-cells contains RTEs, an assay was devised to detect physical evidence of recent TCR gene rearrangement. Focus was made on rearrangements at the β locus because the complete sequence of this locus has been obtained (Rowen, L., et als. (1996) Science 272:1755-1762, “The complete 685-kilobase DNA sequence of the human β T cell receptor locus”), permitting the construction of a panel of Vβ-specific primers to assess the diversity of rearranged TCRs Moreover, allelic exclusion is more complete at the TCRβ locus than at the TQRα locus (Petrie, H. T., et als. (1993) J. Exp. Med. 178:615-622 “Multiple rearrangements in T cell receptor alpha chain genes maximize the production of useful thymocytes;” and Mason, D. (1994) Int. Immunol. 6:881-885 “Allelic exclusion of alpha chains in TCRs”).

Rearrangements at this locus are a salient feature of intrathymic T-cell production and require expression of the recombination activation genes (RAG-1 and RAG-2) and recognition of conserved heptamer and nonamer recombination signal sequences (RSSs) flanking each V, D, and J gene segment (Okazaki, et al. (1987) Cell 49:477-485 “T cell Receptor p Gene Sequences in the Circular DNA of Thymocyte Nuclei: Direct Evidence for Intramolecular DNA Deletion in V-D-J Joining;” Chien, Y., et als. (1984) Nature 309:322-326 “Somatic recombination in a murine T-cell receptor gene;” Malissen, M., et als. (1984) Cell 37:1101-1110 “Mouse T cell antigen receptor, structure and organization of constant and joining gene segments encoding the β polypeptide;” Lewis, S. M. (1994) Adv. Immunol. 56:27-150 “The mechanism of V(D)J joining: lessons from molecular, immunological, and comparative analyses;” and Schatz, D. G., et als. (1989) Cell 59:1035-1048 “The V(D)J recombination activating gene, RAG-1.” As the coding segments are brought together, excision-ligation of the heptamer-heptamer signal joint creates an episomal TCR rearrangement DC (Okazaki, et al. (1987) Cell 49:477-485 “T cell Receptor p Gene Sequences in the Circular DNA of Thymocyte Nuclei: Direct Evidence for Intramolecular DNA Deletion in V-D-J Joining;” Roth, et als. (1992) Cell 70:983-991 “V(D)J recombination: broken DNA molecules with covalently sealed (hairpin) coding ends in SCID mouse thymocytes”), bearing two identifiers: first, each Vβ-Dβ DC has a precise molecular weight determined by the length of intervening, noncoding DNA; secondly, a unique DNA sequence bridges the signal joint. Using the known nucleotide sequences of the non-coding DNA regions adjacent to Vβ2, Vβ17, Vβ5.1 and Dβ1 (Rowen, L., et als. (1996) Science 272:1755-1762, “The complete 685-kilobase DNA sequence of the human β T cell receptor locus”), primers were designed such that a PCR product would only be amplified if they were facing each other within a closed DC, as seen in Table 1.

TABLE 1
Primary sequence of primers required
for βDCs detection/amplification

| Pri- | | SEQ |
|---|---|---|
| mer | | ID |
| name | Nucleoticle sequence | No: |
| DC- | 5′-gcacacacactcccagatgtctcagtcaggaaagc-3′ | 1 |
| Vβ2 | | |
| DC- | 5′-ttftccccagccctgagftgcagaaagcccc-3′ | 2 |
| Vβ5.1 | | |
| DC- | 5-cgtttcctgccatcatagagtgcagaggagccctgt-3′ | 3 |
| Vβ17 | | |
| DC- | 5′-gtcatagcttaaaaccctccgagtgacgcacagcc-3′ | 4 |
| Dβ1 | | |
| Cir- | 5′-ggagggcagctgcaggggftcftgc-3′ | 5 |
| cle- | | |
| Vβ2 | | |
| Cir- | 5-ccacaftgggccagggaggtttgtgc-3′ | 6 |
| cle- | | |
| Vβ5.1 | | |
| Cir- | 5′-gtcggggaagcaggactgggcacatftatgc-3′ | 7 |
| cle- | | |
| Vβ17 | | |

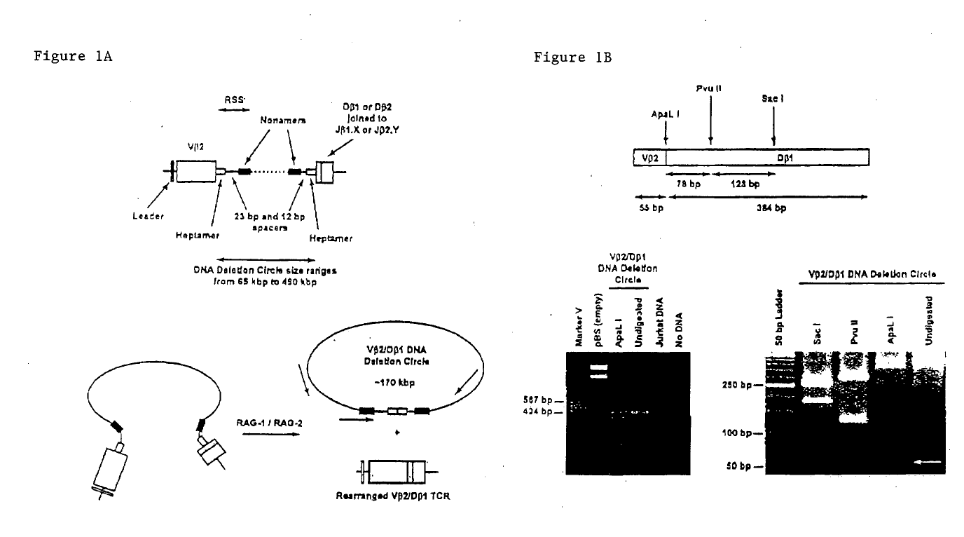

As shown in FIG. 1B, the product amplified for a Vβ2/Dβ1 rearrangement would have a predicted size of 439 bp, with characteristic restriction enzyme sites. In the case of DCs specific for Vβ17/Dβ1 and Vβ5.1/Dβ1 rearrangements, the corresponding molecular weights would be 445 bp and 442 bp, respectively.

The specificity and reliability of this strategy was first assessed in developing human thymocytes expected to have a high frequency of deletion circles (Shortman, K. (1992) Curr. Opin. Immunol. 4:140-146 “Cellular aspects of early T cell development”). DNA was extracted from 2 different samples of human CD4⁺CD8⁺ thymocytes harvested from Thy/Liv organs of SCID-hu mice (Rabin, L., et als. (1996) Antimicrob. Agents Chemother. 40:755-762 “Use of standardized SCID-hu Thy/Liv mouse model for preclinical efficacy testing of anti-human immunodeficiency virus type 1 compounds”). After amplification using the primers specific for Vβ2/Dβ1 DCs, all were found to generate the expected 439 bp PCR product. As shown in FIG. 1B, this product carried predicted restriction enzyme recognition sites for Sac1, Pvu11, and ApaL-1, and was not observed with PCR performed on DNA from Jurkat cells (a Vβ8.1 T-cell line which should not carry Vβ2/Dβ1 DCs). Nucleotide sequence analysis of the PCR product confirmed its identity to the predicted sequence spanning the signal joint of the Vβ2/Dβ1 DC (not shown).

Quantitative Assessment of Cells Having Recently Undergone β Chain TCR Rearrangement

Within a population of cells, the fraction bearing DCs should be proportional to that which has recently undergone TCR rearrangement.

To directly compare this fraction between different cell populations, a semi-quantitative assay was developed to measure a dilution endpoint of DC DNA within a given amount of total cell DNA. DNA was diluted in four replicate series and PCR was carried out to determine whether a given well was positive or negative for the DC PCR product. The “50% DC endpoint”, measured in terms of nanograms of input DNA, was calculated using either the Reed-Muench method (Reed, L. J., et al. (1938) Am. J. Hyg. 27:493-497 “A simple method of estimating fifty percent endpoints;” and Lenette, E. H. (1964) American Public Health Association, New York, p. 45 “General principles underlying laboratory diagnosis of virus and reckettsial infections, in Diagnostic Procedures of Virus and Rickettsial Disease”), or a maximum likelihood estimate (Myers, L. M., et al. (1994) J. Clin. Microbiol. 32:732-739 “Dilution assay statistics”). The 50% DC endpoint represents the median minimal amount of DNA from which a deletion circle may be amplified by nested PCR; the Deletion Circle Frequency (DCF) was arbitrarily defined as the reciprocal of the “50% DC endpoint” (×100) and is proportional to the number of deletion circles which can be amplified from 100 ng of input DNA.

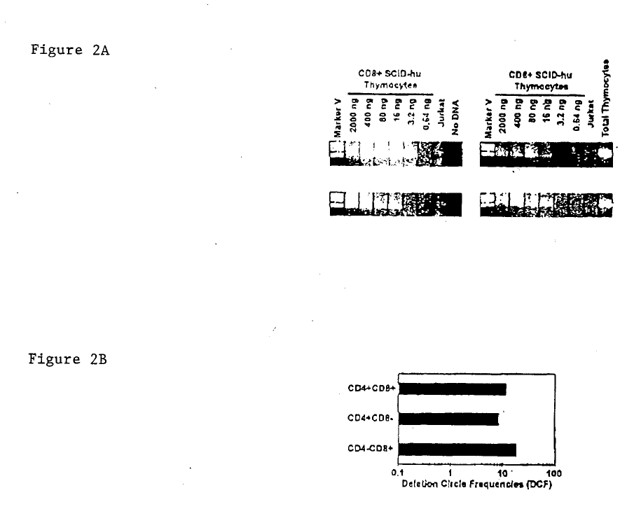

FIG. 2A shows a representative experiment using the assay to quantitate DCs. Four replicate dilution series of DNA from CD3⁺CD8⁺ (single positive, SP) thymocytes were amplified with primers specific for Vβ2/Dβ1 DC, and these yielded a positive PCR signal for deletion circles at final (highest) dilutions of 16, 16, 16, and 3.2 ng input DNA. This corresponds to a 50% DC endpoint of 5.47 ng (as determined by the Reed-Muench method) and a DCF of 18.3 (=100/5.47). Assuming typical recovery of DNA and amplification sensitivity, this would return minimum estimate of 1 DC in 547 SP8 thymocytes or (since 2-5% of total express a Vβ2/Dβ1 TCR) 11-22 Vβ2/Dβ1 SP8 thymocytes.

As may be seen in FIG. 2B, similar frequencies of DC were noted in sorted populations of CD3⁺CD4⁺ and CD4⁺CD8⁺ thymocytes, yielding DCFs of 8.4 and 11.7, respectively.

TCR Vβ Deletion Circles in Circulating Peripheral Blood T-Cells

The Vβ DC assay was used to determine whether Vβ DCs were present in various populations of human peripheral blood T-cells. T-cells in cord blood were examined first.

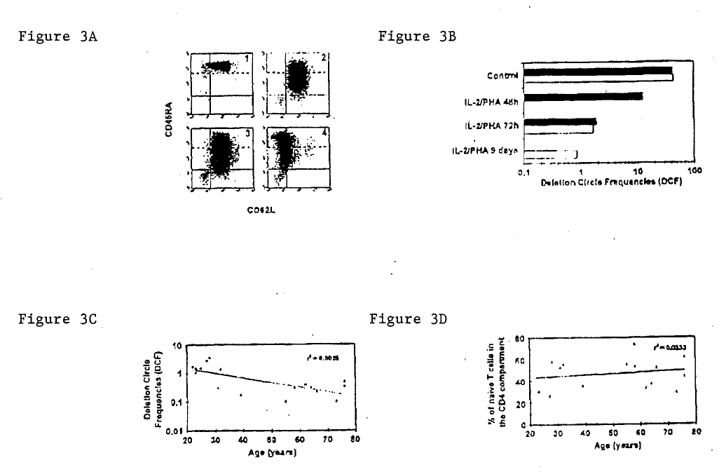

As may be seen in panel 1 of FIG. 3A, flow cytometric analysis revealed that >95% of CD4+T-cells in unstimulated cord blood carried the “naive” CD45RA⁺CD62L⁺ phenotype and all of these cells were “bright” for CD45RA staining.

As may be seen in FIG. 3B, the frequency of DCs within unstimulated cord blood was higher than that observed for single positive thymocytes (with DCFs approximating 43.1 and 41.8 in the two cord blood specimens compared to values of 18.3 and 8.4 for SP8 and SP4 thymocytes, respectively).

As shown in panels 2-4 of FIG. 3A, after 9 days of stimulation in vitro with PHA and IL-2, the percentage of CD4⁺ cord blood T-cells with the “naive” CD45RA^{Bright}CD62L⁺ phenotype dropped to negligible levels and most cells were instead negative for CD62L and/or dimly positive for CD45RA.

As shown in FIG. 3B, within this same time frame, the frequency of DCs dropped from an average of 42.5 DCF to 0.85 DCF, a 50-fold decrease over a 9-day period.

These results indicate that DCs may be detected in circulating T-cells and that their detection is correlated with the presence of cells bearing the “naive” CD45RA⁺CD62L⁺ phenotype.

Inverse Correlation Between Frequencies of Deletion Circles and Age

DCs were then quantitated in the peripheral blood of 17 adult individuals, ranging in age from 22 to 76 years. In each sample, naive CD4⁺ CD45RA⁺CD62L⁺ and memory/effector CD4⁺CD45RO⁺CD62L⁻ cells were quantitated by flow cytometry and sort-purified for determination of DC frequency.

As shown in FIG. 3C, within the population of circulating CD4⁺CD45RA⁺CD62L⁺ T-cells, DCs were observed with a frequency that was higher than that found in the CD4⁺CD45RO⁺CD62L⁻ population, which had nondetectable levels of DC in these 17 individuals (not shown).

FIG. 3C shows that as a function of age, there was a consistent decrease in the frequency of DCs within the CD4⁺CD45RA⁺CD62L⁺ sub-population (r²=0.5026, p=0.0045), even though individuals across this age range had equivalent percentages of CD45RA⁺CD62L⁺ within their CD4⁺ T-cells; as shown in FIG. 3D R²=0.0233, p=0.5123).

These data suggest that RTEs exist within the circulating population of CD4⁺CD45RA⁺CD62L⁺ T-cells of adults and that their proportion decreases with age.

The DC assay provides a much more reliable estimate of de novo generated T-cells than that provided by phenotypic cell-surface markers such as CD45RA and CD62L.

Detection of Deletion Circles in Other T-Cell Populations

To determine whether other sub-populations of circulating CD4⁺ T-cells might harbor TCRβ rearrangement DCs, cells were sort-purified into CD4⁺CD45RA⁺CD62L⁺, CD4⁺CD45RO⁺CD62L⁻, CD4⁺CD45RO⁺CD62L⁺, and CD4⁺CD45RO⁻CD62L⁺ sub-populations. In eight individuals ranging in age between 22 and 76 years, the highest frequency of DC was found in the CD45RA⁺ CD62L⁺ sub-populations and the lowest in the CD45RO⁺CD62L⁻ sub-population, as shown in Table 2

TABLE 2
DCF values for multiple TCR VβDβ rearrangements
in FACS-sorted sub-populations
of CD4^{+ T-cell sub-populations}

| | Age | | | | | | | | |
|---|---|---|---|---|---|---|---|---|---|
| | 22 | 23 | 25 | 28 | 31 | 32 | 39 | 76a | 76b |
| %^{a} | | | | | | | | | |
| CD4⁺CD45RA⁺CD62L⁺ | 32 | 30 | 25 | 36 | 52 | 59 | 35 | 44 | 62 |
| CD4⁺CD45RO⁺CD62L⁺ | 18 | 25 | 28 | 42 | 38 | 25 | 18 | 43 | 33 |
| CD4⁺CD45RO⁺CD62L⁺ | 39 | 19 | 32 | 13 | 9 | 10 | 23 | 6 | 4 |
| % TCR Vβ2^{b} | N.D. | N.D. | 8 | N.D. | 6 | N.D. | N.D. | 9 | 9 |
| DCF (Vβ2/Dβ1 DC)^{c} | | | | | | | | | |
| CD4⁺CD45RA⁺CD62L⁺ | 1.67 | 10.12 | 1.46 | 3.63 | 0.50 | 1.37 | 0.17 | 0.50 | 0.33 |
| CD4⁺CD45RO⁺CD62L⁺ | 0.50 | N.D. | 0.73 | N.D. | 0.75 | <0.1 | N.D. | <0.1 | 0.1 |
| CD4⁺CD45RO⁺CD62L⁻ | 0.22 | <0.07 | <0.1 | <0.1 | <0.1 | <0.1 | <0.1 | <0.1 | N.D. |
| DCF (Vβ5.1/Dβ1 DC)^{c} | | | | | | | | | |
| CD4⁺CD45RA⁺CD62L⁺ | N.D. | N.D. | N.D. | N.D. | 0.29 | 7.97 | N.D. | N.D. | 0.71 |
| DCF (Vβ17/Dβ1 DC)^{c} | | | | | | | | | |
| CD4⁺CD45RA⁺CD62L⁺ | N.D. | 1.72 | N.D. | 0.85 | 1.12 | N.D. | 0.37 | N.D. | N.D. |

^{a}Frequency of each sort-purified sub-population within CD4⁺ T-cell;
^{b}Flow cytometry-derived percentages of TCRVβ2+ T-cells found in the CD4^{+ T-cell population;}
^{c}DCs from different Vβ rearrangements were quantitated in the naive (CD45RA⁺CD62L⁺) and memory effector (CD4SRO⁺CD62L⁻ and CD45RO⁺CD62L⁺) CD4⁺ T-cell sub-populations of healthy adults, as described. The frequency of TCRVβ2⁺CD4⁺ T-cells (b) remains quite constant with increasing age while the corresponding DCF trend (c) decreases.
ND not done.

DCs were also found in the CD45RO⁺CD62L⁺ sub-population in 4 out of 8 individuals tested, albeit at a lower frequency. Finally, DCs were detected in T-cells with the phenotype CD45RO⁻CD62L⁺ (not shown) and CD45RO⁺CD62L⁻, although only one out of 9 individuals showed detectable levels of DCs in the latter compartment. These cells may possibly represent direct progeny of RTEs in the CD45RA⁺CD62L⁺ sub-population; alternatively, DCs may be present within them as a consequence of extrathymic TCR rearrangements (Mackall, et al. (1997) Immunol. Today 18: 245-251 “T-cell Regeneration: All Repertoires are not Created Equal; and Garcia-Ojeda, M. E., et als. (1998) J. Exp. Med. 187:1813-1823 “An alternate pathway for T cell development supported by the bone marrow microenvironment: recapitulation of thymic maturation”).

The TCR repertoire in RTEs is at least oligoclonal. Previous studies have demonstrated the presence but not the degree of TCR diversity of RTEs in adult humans (Scollay, et als. (1980) Eur. J. ImmunoL 10:210-218 “Thymus cell migration. Quantitative aspects of cellular traffic from the thymus to the periphery in mice;” and Jamieson, B. D., et als. (1999) Immunity, 10:569-575 “Generation of functional thymocytes in the human adult”).

To address this parameter of diversity, primers were generated which could amplify DCs issued from three different TCRVβ-Dβ rearrangements (Vβ2/Dβ1, Vβ5.1/Dβ1, and Vβ17/Dβ1). Flow cytometric analyses (not shown) revealed different percentages of circulating T-cells bearing these three Vβs (Vβ2=8-10%; Vβ5.1=3-4%; Vβ17=3-4%). Results illustrated in Table 2 clearly show that DCs detectable in circulating human T-cells encompass several (at least two) Vβs and were present not only in the CD45RA⁺CD62L⁺ but also in the CD45RO⁺CD62L⁺ sub-populations of CD4⁺ T-cells.

Interestingly, the relative frequency of DCs from different Vβ regions did not correlate with the proportion of peripheral blood lymphocytes (PBLs) expressing these TCR Vβ products. For instance, Vβ2⁺ T-cells were always at least two fold more abundant in PBLs from normal individuals compared to Vβ5.1⁺ or Vβ17⁺ T-cells (not shown). Yet, analysis of DCF values shown in Table 2 indicate that, in the two individuals tested (aged 31 and 32 years), Vβ5.1/Dβ1 or Vβ17/Dβ1 DCs were 2- to 5-fold more abundant than Vβ2/Dβ1 DCs. These differences in the relative abundance of Vβ-DCs compared to the expected frequencies of their parental cell populations could reflect both a relative dilutional effect on some Vβ-DCs due to varying degrees of peripheral expansion in Vβ-specific subsets, as well as a relative overestimate of some sub-populations due the detection of DCs from non-productive rearrangements that might be more prevalent in certain Vβ subsets.

In sum, these experiments demonstrate that TCRβ DCs can be detected within thymocytes and within circulating human CD4⁺ T-cells with a “naive” (CD45RA⁺CD62L⁺) phenotype. Detection of such DCs is specific, reliable, and quantitative. The DCs are generated upon rearrangement of multiple Vβ coding segments. Finally, DCs in CD4⁺CD45RA⁺CD62L⁺ T-cells are observed in a pattern which is consistent with known parameters of intrathymic maturation: their frequency decreases as cord blood T-cells are stimulated to divide in vitro and in older individuals who have less abundant thymus, as measured in autopsy series or by non-invasive radiography. As such, quantitation of DCs within human peripheral blood CD4⁺CD45RA⁺CD62L⁺ T-cells represents a measure of RTEs and, hence, thymic function.

These results confirm previous inferences about thymic function. First, the finding of DCs within the CD4⁺CD45RA⁺CD62L⁺ population of adult individuals aged 23-76 years underscores the premise that the thymus, though less functional, is nonetheless operative into adulthood (McCune, J. M. (1997) Sem. Immunol. 9:397-404 “Thymic Function in HIV-1 Disease;” Steinmann, G. G. (1986) Histopathology and Pathology (Muller-Hermelink H. K., ed) Springer, New York, pp 43-48 (1986), “Changes in the human thymus during aging, in The Human Thymus;” McCune, et als. (1998) J. Clin. Invest. 101:2301-2308 “High prevalence of thymic tissue in adults with human immunodeficiency virus-i infection;” Douek, et als. (1998) Nature 396:690-695 “Changes in thymic function with age and during the treatment of HIV infection;” Jamieson, B. D., et als. (1999) Immunity, 10:569-575 “Generation of functional thymocytes in the human adult”). Secondly, the fact that the frequency of DCs decreases in the CD4⁺CD45RA⁺CD62L⁺ population as a function of age demonstrates that this population is heterogeneous (Tough, D F, et al. (1995) Stem Cells. 13:242-249 “Life span of naive and memory T cells;” and Bell, E. B., et al. (1990) Nature 348:163-166 “Interconversion of CD45R subsets of CD4 T cells in vivo”), and that its composition is age-dependent. It may not be useful, in other words, to assume that the presence (or reappearance) of such cells is synonymous with “immune reconstitution” (Autran, B., et als. (1997) Science 277:112-116 “Positive effects of combined antiretroviral therapy on CD4⁺ T cell homeostasis and function in advanced HIV disease;” Fleury, S., et als. (1998) Nat. Med. 4:794-801 “Limited CD4+T-cell renewal in early HIV-1 infection: effect of highly active antiretroviral therapy;” Pakker, N. G., et als. (1998) Nat. Med. 4:208-214 “Biphasic kinetics of peripheral blood T cells after triple combination therapy in HIV-1 infection: a composite of redistribution and proliferation;” Komanduri, K. V. et als. (1998) Nat. Med. 4:953-956 “Restoration of cytornegalovirus-specific CD4+T-lymphocyte responses after ganciclovir and highly active antiretroviral therapy in individuals infected with HIV-1”) Finally, the finding of DCs within other populations of circulating T-cells raises the possibility that extrathymic sources (e.g. gut or liver) may contribute to formation of the circulating TCR repertoire (Mackall, et al (1997) Immunol. Today 18:245-251 “T-cell Regeneration: All Repertoires are not Created Equal;” and Garcia-Ojeda, M. E., et als. (1998) J. Exp. Med. 187:1813-1823 “An alternate pathway for T cell development supported by the bone marrow microenvironment: recapitulation of thymic maturation”).

The present invention will be more readily understood by referring to the following examples which are given to illustrate the invention rather than to limit its scope.

### EXAMPLE I

### Isolation of Thymocytes

Methods for maintenance of SCID-hu mice and harvest of thymocytes from SCID-hu Thy/Liv organs were identical to those previously published (Rabin, L., et als. (1996) Antimicrob. Agents Chemother. 40:755-762 “Use of standardized SCID-hu Thy/Liv mouse model for preclinical efficacy testing of anti-human immunodeficiency virus type 1 compounds”). In some cases, SCID-hu Thy/Liv organs were harvested and placed in RPM1 1640 media (Life Technologies) supplemented with 10% fetal calf serum (FCS) (Summit Biotechnology, Fort Collins, Colo.) and transported overnight at 4° C. prior to harvest of thymocytes. Following isolation, thymocytes were resuspended in phosphate buffered saline (PBS) supplemented with 2% FCS and kept on ice prior to staining with monoclonal antibodies for flow cytometric analysis or cell sorting. All procedures and practices were approved by the University of Calif., San Francisco Committee on Human Research (CHR) or the University of California, San Francisco Committee on Animal Research

Isolation of Peripheral Blood Mononuclear Cells (PBMC)

Whole blood samples from human subjects were collected by phlebotomy into EDTA collection tubes (Becton Dickinson). Peripheral blood mononuclear cells (PBMC) were isolated from whole blood by density gradient centrifugation (Life Technologies). PBMC were washed twice with PBS before resuspension in PBS supplemented with 2% FCS prior to staining with monoclonal antibodies for flow cytometry or cell sorting.

Stimulation of Cord Blood Cells in vitro

Human umbilical cord blood cells were obtained (with CHR approval) from healthy delivery specimens and placed in heparinized collection tubes (Becton Dickinson) under sterile conditions. Cord blood mononuclear cells (CBMCs) were isolated as described above for whole blood specimens and resuspended at a concentration of 2×10⁶ cells/ml in RPMI 1640 supplemented with 10% human AB serum (Ultraserum, Gemini Bio-Products). CBMCs were then cultured (at 37° C. in 5% CO₂ for 48 hr, 72 hr, 96 hr, or 9 days (time points encompassed in 2 different experiments) and stimulated with 5 ug/ml of phytohemagglutinin (PHA) (Sigma) and 10 U/ml purified interleukin-2 (IL-2) (Boehringer Mannheim). The supplemented medium was changed every 3 days. Cell culture controls did not receive PHA or IL-2 stimulation but were cultured for 72 hr in the same medium. Aliquots of the cell cultures at different time points were analyzed by flow cytometry for the expression of the cell surface markers, CD45RA and CD62L.

### EXAMPLE II

### Immunophenotypic Analysis and Cell Sorting by Flow Cytometry

PBMC, thymocytes from SCID-hu mice, or CBMC were stained with fluorescent-conjugated monoclonal antibodies specific for cell surface markers at a concentration of 107 cells/ml at 40° C. for 30 minutes. Following staining, cells were washed with PBS supplemented with 2% FCS and sorted either on a FACStar or a FACS Vantage cell sorter (both from Becton Dickinson). The cells were stained with one of the following antibody combinations: 1) anti-CD8-FITC (Becton Dickinson) and anti-CD4-PE (Becton Dickinson); 2) anti-CD45RA-FITC (Immunotech) or anti-CD45RO-FITC (Immunotech), anti-CD62L-PE (Becton Dickinson), and anti-CD4-ECD (Coulter); 3) anti-CD62L-FITC (Pharmingen), anti-CD45RA-PE (Pharmingen) and anti-CD4-TC or anti-CD4-APC (Caltag) Sort purities were checked after each sort and were not less than 97%. For analysis of cord blood CD45RA and CD62L expression, CBMC were stained with anti-CD45RA-FITC (Immunotech) and anti-CD62L-PE (Becton Dickinson) and analyzed using a FACScan® cytometer and Cell Quest software (both from Becton Dickinson).

### EXAMPLE III

Detection of TCR β Rearrangement Deletion Circles

Total DNA from distinct cell populations was extracted and purified via a standard protocol (Ausubel, F. M. et al. (1987) Interscience, New York, pp 2.2.1-2.2.3 Current Protocols in Molecular Biology, “Preparation of genomic DNA from mammalian tissue”) before spectrophotometric quantitation at 260 nm and 280 nm. The freshly isolated DNA was stored at 4° C. for further processing. Thermal cycling was performed for 30 cycles (1 min at 94° C., 1 min 30 sec at 65° C., 1 min 30 sec at 72° C.) for each round of a semi-nested PCR protocol designed to detect VPDP-specific deletion circles generated by TCRβ recombination. All first and second round primers were generated to fully hybridize with non-coding regions of the TCRβ locus (Rowen, L., et als. (1996) Science 272:1755-1762, “The complete 685-kilobase DNA sequence of the human β T cell receptor locust) located next to the recombination signal sequences (RSSs) (GeneBank accession numbers U66059, U66060, and U66061), as shown in Table 1. Four PCR replicates were done on each total DNA serial dilution to ensure a precise read-out for each experiment. Concentrations of total DNA were adjusted so that a constant volume of 3 ul was added to each 50 ul PCR reaction [200 uM dNTPs, 1× PCR buffer (Boehringer Mannheim), 100 ng of each primers and 2 U of Taq polymerase (Boehringher Mannheim)]. From the first PCR amplification, 3 ul were used as template for the second (semi-nested) PCR reaction (same conditions) using the “Circle” primer and the DC-Dβ1 primer.

### EXAMPLE IV

### Quantitative Analysis of Endpoint Dilutions

Second-round PCR products were visualized with ethidium bromide on 1.25% agarose gels and digitally photographed. Individual amplifications were scored as positive or negative by two observers. The highest dilution returning a positive amplification was taken as the endpoint for each dilution series. Dilution series with greater than two “skipped” well (a failed amplification followed by a successful amplification at higher dilution) were omitted from the analysis. The abundance of deletion circles was estimated by the method of Reed-Muench (Reed, L. J., et al. (1938) Am. J. Hyg. 27:493-497 “A simple method of estimating fifty percent endpoints”); Lenette, E. H. (1964) American Public Health Association, New York, p. 45 “General principles underlying laboratory diagnosis of virus and reckettsial infections, in Diagnostic Procedures of Virus and Rickettsial Disease”). This method uses information from replicate dilution series to estimate an endpoint (measured in terms of ng input DNA) in which 50% of samples were positive for DC (the 50% DC endpoint). The Deletion Circle Frequency (DCF) was arbitrarily defined as the reciprocal of the “50% DC endpoint” (×100).

Alternatively, the semi-nested PCR data were analyzed by a maximum likelihood estimated method of dilution endpoint with a parametric method (Myers, L. M., et al. (1994) J. Clin. Microbiol. 32:732-739 “Dilution assay statistics”). Unlike the Reed-Muench method, this method returns an estimate of goodness of fit of the data to the estimated endpoint. Endpoints estimated by the two methods were highly correlated (r²=0.929) and the choice of method did not alter the conclusions drawn from the data. The degree of inter- and intra-assay variation was assessed by performing two independent experiments on two different samples from the same individuals (n=3) and ranged on the order of 2-3 fold (data not shown).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for detecting recent thymic emigrant (RTE) diversity in a T-cell sub-population of a patient, said method comprising:
a) isolating a T-cell sub-population from a patient;
b) extracting genomic DNA from said T-cell sub-population;
c) amplifying said genomic DNA with a primer specific for a T-cell receptor β chain DNA rearrangement deletion circle (TCRβDC) family; and
d) detecting said TCRβDC in said amplified DNA, said TCRβDC being indicative of the presence of a RTE.

2. A method according to claim 1, wherein said extracted genomic DNA is diluted prior to said amplification.

3. A method according to claim 2, wherein said amplification is effected with a polymerase chain reaction (PCR).

4. A method according to claim 3, wherein said extracted genomic DNA is spectrophotometrically quantitated to detect said TCRβDC prior to said dilution.

5. A method according to claim 4, wherein said dilution is effected 4 or 5 folds.

6. A method according to claim 5, wherein a dilution endpoint of TCRβDC is determined for said dilution.

7. A method according to claim 6, wherein a positive signal corresponding to an endpoint is detected at a highest dilution, and wherein a TCRβDC 50% endpoint and a TCRβDC frequency are determined.

8. A method according to claim 7, wherein said 50% endpoint is calculated with a Reed-Muench method or a maximum likelihood estimate.

9. A method according to claim 8, wherein said extracted genomic DNA is amplified a first time with a Dβ-specific primer and a Vβ-specific primer, said amplified DNA being amplified a second time with a nested primer.

10. A method according to claim 9, wherein said TCRβDC is detected with an agarose gel electrophoresis under a UV light.

11. A method according to claim 10, wherein said primer has a sequence selected from the group consisting of SEQ ID NO:1, SEQ ID NO:2, SEQ ID NO:3, SEQ ID NO:4, SEQ ID NO:5, SEQ ID NO:6 and SEQ ID NO:7.

12. A method according to claim 11, wherein said T-cell sub-population is selected from the group consisting of CD3⁺CD8⁺ thymocytes, CD4⁺CD8⁺ thymocytes, CD3⁺CD4⁺CD8⁻ thymocytes, CD3⁺CD4⁻CD8⁺ thymocytes, CD4⁺CD45RA⁺CD62L⁺ lymphocytes, CD4⁺CD45RA⁺CD62L⁻ lymphocytes, CD4⁺CD45RO⁺CD62L⁺ lymphocytes, CD4⁺CD45RO⁺CD62L⁻ lymphocytes and CD4⁺CD56RO⁻CD62L⁺ lymphocytes.

13. A method according to claim 12, wherein said T-cell sub-population is isolated from a peripheral blood sample, a cord blood sample or a tissue section collected from said patient.

14. A method according to claim 13, wherein said amplified DC family comprises a Vβ/Dβ DC family.

15. A method according to claim 14, wherein said primer is specific for a Vβ2/Dβ1, Vβ5.1/Dβ1, Vβ9/Dβ1, Vβ14/Dβ1, Vβ16/Dβ1, Vβ17/Dβ1 or Vβ22/Dβ1 DC family.

16. A method according to claim 15, wherein said specifice primer is used with TaqMan.

17. A method according to claim 15, wherein said cell-surface marker comprises CD45RA and CD62L.

18. A method according to claim 17, wherein said patient is infected with HIV or has undergone a myeloablation.

19. A method according to claim 18, wherein said T-cell sub-population is isolated from said peripheral blood sample by staining said sample with fluorescent-conjugated monoclonal antibodies specific for a cell-surface marker.

20. A method according to claim 18, wherein said T-cell sub-population is isolated by flow cytometry.

21. A method according to claim 18, wherein said T-cell sub-population is isolated by sort-purification.

22. A method according to claim 21, wherein said genomic DNA is recovered with a proteinase K.

23. A method for developing monoclonal antibodies to identify a recent thymic emigrant subset in a patient.

24. A method for detecting T-cell receptor β chain DNA rearrangement deletion circles (TCRβDCs) in a cell population from a patient, said method comprising:
a) isolating a cell population from a patient;
b) extracting genomic DNA from said cell population;
c) diluting said extracted DNA;
d) amplifying said diluted DNA with a primer specific for a TCRβDC family; and
e) effecting an endpoint dilution analysis of said amplified DNA for said TCRβDC family.

25. A method for detecting T-cell receptor β chain DNA rearrangement deletion circles (TCRβDC) in a T-cell, said method comprising:
a) amplifying a genomic DNA of said cell with a primer specific for a TCRβDC family; and
b) detecting said amplified DNA indicative of a newly generated T-cell.
